(19) United States
(12) Patent Application Publication
(10) Pub. No.: US 2007/0249557 A1
(43) Pub. Date: Oct. 25, 2007

(54) Compositions and methods for viscosupplementation

(75) Inventor: Gregory D. Jay, Norfolk, MA (US)

Correspondence Address: CLARK & ELBING LLP, 101 FEDERAL STREET, BOSTON, MA, 02110, US

(73) Assignee: Mucosal Therapeutics, LLC, Wellesley, MS (US)

(21) Appl. No.: 11/784,049

(22) Filed: Apr. 5, 2007

Related U.S. Application Data
(63) Continuation-in-part of application No. 11/658,233.
(60) Provisional application No. 60/590,766, filed on Jul. 23, 2004.

(51) Int. Cl. A61K 31/728

(52) U.S. Cl. 514/54

(57) ABSTRACT

The invention provides viscosupplementation compositions that include hyaluronic acid, or a polymer thereof and a tribonectin, or an analog, derivative, or fragment thereof. Such compositions are useful for the lubrication and chondroprotection of mammalian joints.

### CROSS-REFERENCE TO RELATED APPLICATIONS

The application is a continuation-in-part of U.S. Ser. No. 11/658,233, filed Jan. 23, 2007, pending, which is a National stage of PCT/US2005/026004, filed Jul. 22, 2005, which claims the benefit of U.S. Provisional Application No. 60/590,766, filed Jul. 23, 2004, each of which is incorporated herein by reference.

### BACKGROUND OF THE INVENTION

The invention relates to lubrication of mammalian joints.

Osteoarthritis (OA) is the one of the most common forms of joint disease. Factors which contribute to the development of OA include a family history of OA, previous damage to the joint through injury or surgery, and age of the joint, i.e., “wear and tear” of the articulating surfaces of the joint. OA is very common in older age groups, but can affect children as well.

Current treatment is directed to relieving pain and other symptoms of OA, e.g., by administering analgesics and anti-inflammatory drugs. Other therapeutic approaches include viscosupplementation by administering hyaluronic acid (HA) and derivatives thereof to joint tissue to increase the viscosity of synovial fluid. Despite the useful properties of HA, such as biocompatibility, (bio)degradability, resorption, non-immunogenicity, very low and rare pyrogenicity, it is a highly viscous material, with poor lubricating properties. Still needed are improved methods and compositions for viscosupplementation.

### SUMMARY OF THE INVENTION

Accordingly, in a first aspect, the present invention features a viscosupplementation composition that includes hyaluronic acid (HA), or a polymer thereof, in a concentration in the range of from about 0.1 mg/mL to about 50 mg/mL and a tribonectin, or analog, derivative, or fragment thereof, at a concentration of from 0.1 μg/mL to 1.0 mg/mL. In an embodiment, the HA, or a polymer thereof, is present in a concentration in the range of from about 1.0 mg/mL to about 5 mg/mL, more preferably in a concentration in the range of about 1.0 mg/mL to about 2.5 mg/mL. In other embodiments, the HA is present in the compositions of the invention at a concentration in the range of from about 2.5 mg/mL to about 5.0 mg/mL, a concentration in the range of from about 3.0 mg/mL to about 4.0 mg/mL, or a concentration in the range of from about 10.0 mg/mL to about 25.0 mg/mL. In other embodiments, the HA is present in the compositions at a concentration of between about 0.1 mg/mL to about 0.99 mg/mL or at a concentration of between about 5.1 mg/mL to about 50 mg/mL.

In yet another embodiment, the tribonectin, or analog, derivative, or fragment thereof, is present in a concentration in the range of from about 10 μg/mL to about 500 μg/mL, more preferably in a concentration in the range of about 1.0 μg/mL to about 250 μg/mL. In other embodiments, the tribonectin, or analog, derivative, or fragment thereof, is present in a concentration in the range of from about 100.0 μg/mL to about 1.0 mg/mL. In other embodiments, the tribonectin is present in the compositions at a concentration of between about 0.11 g/mL to about 9.9 μg/mL or at a concentration of between about 250.1 μg/mL to about 1.0 mg/mL.

In another embodiment, a HA/tribonectin composition of the invention includes hyaluronic acid and tribonectin at a molar ratio of from about 2:1 to about 4:1, respectively. In another embodiment, the hyaluronic acid and tribonectin are present in the compositions of the invention at a molar ratio of from about 10:1 to about 50:1, respectively. In another embodiment, the hyaluronic acid and tribonectin are present in the compositions of the invention can be prepared at a molar ratio of from about 100:1 to about 500:1, respectively.

In an embodiment, the HA contains cross-links. In yet another embodiment, the HA is not crosslinked.

In another embodiment, the HA is isolated from a natural source, is produced in vitro, or is chemically synthesized. In yet another embodiment, the tribonectin is isolated from a natural source, is produced recombinantly, or is chemically synthesized.

In another aspect, the invention features a method of lubricating a mammalian joint by contacting the joint with a composition of the invention. The mammal is preferably a human, horse, dog, ox, donkey, mouse, rat, guinea pig, cow, sheep, pig, rabbit, monkey, or cat, and the joint is an articulating joint such as a knee, elbow, shoulder, hip, or any other weight-bearing joint. The compositions of the present invention can be administered, e.g, intra-articularly, or by any other methods known in the art, as is discussed in detail below.

In yet another aspect, the invention features a method of increasing the elasticity of a viscosupplement for the lubrication and chondroprotection of a mammalian joint by adding a tribonectin, or an analog, derivative, or fragment thereof, to the viscosupplement. In an embodiment, the elasticity of the viscosupplement is increased by at least 5%, more preferably at least 10%, 20%, or 30%, and most preferably by at least 40%, 50%, 60%, 70%, or 80% or more. Elasticity of the viscosupplement can be determined according to the methods described in, e.g., U.S. Pat. No. 6,890,901, which is incorporated herein by reference. In an embodiment, the viscosupplement also includes hyaluronic acid. The mammal is preferably a human, horse, dog, ox, donkey, mouse, rat, guinea pig, cow, sheep, pig, rabbit, monkey, or cat, and the joint is an articulating joint such as a knee, elbow, shoulder, hip, or any other weight-bearing joint. The viscosupplement can be administered, e.g., intra-articularly. Alternatively, the mammalian joint can be treated first with a viscosupplement and then subsequently treated separately with the tribonectin, which is added to the viscosupplement in vivo. In an embodiment, the tribonectin, or analog, derivative, or fragment thereof, is present in a concentration in the range of from about 10 μg/mL to about 500 μg/mL, more preferably in a concentration in the range of about 1.0 μg/mL to about 250 μg/mL. In other embodiments, the tribonectin, or analog, derivative, or fragment thereof, is present in a concentration in the range of from about 100.0 μg/mL to about 1.0 mg/mL. In another embodiment, a HA/tribonectin composition of the invention includes hyaluronic acid and tribonectin at a molar ratio of from about 2:1 to about 4:1, respectively. In another embodiment, the hyaluronic acid and tribonectin are present in the compositions of the invention at a molar ratio of from about 10:1 to about 50:1, respectively. In yet another embodiment, the addition of a tribonectin to a viscosupplement (e.g., a viscosupplement containing HA) reduces the viscosity of the viscosupplement by, e.g., at least 10%, more preferably by at least 20%, 30% or 40%, and most preferably by at least 50%, 60%, 70%, or 80% or more. Viscosity of the viscosupplement can be determined according to the methods disclosed in, e.g., U.S. Pat. No. 4,920,104, which is incorporated herein by reference.

In yet another aspect of the invention, the compositions of the invention can be administered to a mammal (e.g., a human) to treat or reduce the symptoms associated with soft tissue injuries, structural injuries, and degenerative or congenital conditions. In particular, the compositions of the invention can be administered to a mammal to treat or to alleviate, inhibit, or relieve the symptoms of osteoarthritis (which includes erosive osteoarthritis and is also known as osteoarthrosis or degenerative joint disease or DJD), rheumatoid arthritis, juvenile rheumatoid arthritis, spondyloarthropathies, gouty arthritis, infectious arthritis, structural joint defects (e.g., torn menisci and cruciate ligaments), traumatic synovitis, and repetitive stress syndromes, as well as inflammation and symptoms associated with Sjogren's syndrome, Crohn's disease, and psoriatic arthritis, and systemic lupus erythematosus.

In another embodiment, the compositions of the invention can be administered to a mammal to alleviate, inhibit, relieve, or treat arthritic conditions associated with spondylitis, including ankylosing spondylitis, reactive arthritis (Reiter's syndrome), arthritis associated with chronic inflammatory bowel disease and AIDS-related seronegative spondyloarthropathy.

In another embodiment, the compositions of the invention can be administered to a mammal to alleviate or inhibit symptoms of rheumatic disease and disorders, e.g., systemic sclerosis and forms of scleroderma, polymyositis, dermatomyositis, necrotizing vasculitis and other vasculopathies, hypersensitivity vasculitis (including Henoch-Schonlein purpura), Wegener's granulomatosis, Giant cell arteritis, mucocutaneous lymph node syndrome (Kawasaki disease), Behcet's syndrome, Cryoglobulinemia, juvenile dermatomyositis, Sjogren's syndrome, overlap syndromes (includes mixed connective tissue disease), polymyalgia rheumatica, erythema nodosum, relapsing polychondritis, tendonitis (tenosynovitis), Bicipital tendenitis, bursitis, Olecranon bursitis, adhesive capsulitis of the shoulder (frozen shoulder) trigger finger, and Whipple's disease.

The compositions of the invention can also be administered to alleviate or inhibit the symptoms of diseases associated with rheumatic states, including, e.g., gout, pseudogout, chondrocalcinosis, amyloidosis, scurvy, specific enzyme deficiency states (including Fabry's disease, alkaptonuria, ochonosisi, Lesch-Nyhan syndrome, and Gaucher's disease), hyperlipoproteinemias (types II, IIa, IV), Ehlers-Danlos syndrome, Marfan's syndrome, pseudoxanthoma elasticum, and Wilson's disease.

The compositions of the invention can be administered to the joint (e.g., the knee, shoulder, wrist, ankle, or elbow) or to connective tissue of a mammal.

As described in U.S. Pat. No. 6,743,774, a tribonectin is an articular boundary lubricant which contains at least one repeat of an amino acid sequence which is at least 50% identical to KEPAPTT (SEQ ID NO:3). A tribonectin is formulated for administration to a mammalian joint. Preferably, the tribonectin is isolated from a natural source, or is a recombinant or chemically-synthesized lubricating polypeptide. For example, a tribonectin includes a substantially pure polypeptide the amino acid sequence of which includes at least one but less than 76 subunits. Each subunit contains at least 7 amino acids (and typically 10 or fewer amino acids). The amino acid sequence of each subunit is at least 50% identical to SEQ ID NO:3, and a non-identical amino acid in the reference sequence is a conservative amino acid substitution. For example, one or both of the threonine residues are substituted with a serine residue. Preferably, the amino acid sequence of the subunit is identical to SEQ ID NO:3. The tribonectin may also contain one or more repeats of the amino acid sequence XXTTTX (SEQ ID NO:4).

Polypeptides or other compounds described herein are said to be “substantially pure” when they have been separated from at least 60% to 75% or more of the components that naturally accompany them. Preferably, polypeptides or other compounds described herein are substantially pure when they are separated from at least about 85 to 90% of the components that naturally accompany them, more preferably at least about 95%, and most preferably about 99% or more. Normally, purity is measured on a chromatography column, polyacrylamide gel, or by HPLC analysis.

Where a particular polypeptide is said to have a specific percent identity to a reference polypeptide of a defined length, the percent identity is relative to the reference polypeptide. Thus, a peptide that is 50% identical to a reference polypeptide that is 100 amino acids long can be a 50 amino acid polypeptide that is completely identical to a 50 amino acid long contiguous portion of the reference polypeptide. It can also be a 100 amino acid long polypeptide which is 50% identical to the reference polypeptide over its entire length.

A polypeptide which is “substantially identical” to a given reference polypeptide or nucleic acid molecule is a polypeptide having a sequence that has at least 85%, preferably 90%, and more preferably 95%, 98%, 99% or more identity to the sequence of the given reference polypeptide sequence or nucleic acid molecule. The term, “identity” has an art-recognized meaning and is calculated using well known published techniques, e.g., Computational Molecular Biology, 1988, Lesk A. M., ed., Oxford University Press, New York; Biocomputing: Informatics and Genome Projects, 1993, Smith, D. W., ed., Academic Press, New York; Computer Analysis of Sequence Data, Part I, 1994, Griffin, A. M. and Griffin, H. G., eds., Humana Press, New Jersey; Sequence Analysis in Molecular Biology, 1987, Heinje, G., Academic Press, New York; and Sequence Analysis Primer, 1991, Gribskov, M. and Devereux, J., eds., Stockton Press, New York).

A tribonectin is characterized as reducing the coefficient of friction (tt) between bearing surfaces. For example, reduction of friction is measured in vitro by detecting a reduction in friction in a friction apparatus using latex:glass bearings. The effect of a tribonectin on joint lubrication can also be determined by measuring an increase in mobility. Tribonectins of the invention are lubricating substances or components of compositions. Polypeptides that have at least 50% (but less than 100%) amino acid sequence identity to a reference sequence are tested for lubricating function by measuring a reduction in the g between bearing surfaces.

A tribonectin may include an O-linked oligosaccharide, e.g., an N-acetylgalactosamine and galactose in the form β(1-3)Gal-GalNAC. For example, KEPAPTT (SEQ ID NO:3) and XXTTTX (SEQ ID NO:4) repeat domains are glycosylated by β(1-3)Gal-GalNAC (which may at times be capped with NeuAc in the form of β(1-3)Gal-GalNAC-NeuAc. The term “glycosylated” with respect to a polypeptide means that a carbohydrate moiety is present at one or more sites of the polypeptide molecule. For example, at least 10%, preferably at least 20%, more preferably at least 30%, and most preferably at least 40% of the tribonectin is glycosylated. Up to 50% or more of the tribonectin can be glycosylated. Percent glycosylation is determined by weight.

A tribonectin can contain a substantially pure fragment of megakaryocyte stimulating factor (MSF). For example, the molecular weight of a substantially pure tribonectin having an amino acid sequence of a naturally-occurring tribonectin is in the range of, e.g., about 206-345 kDa, more preferably about 220-280 kDa. Preferably, the apparent molecular weight of a tribonectin is less than 230 kDa, more preferably less than 250 kDa, and most preferably less than 280 kDa. A protein or polypeptide fragment is defined as a polypeptide which has an amino acid sequence that is identical to part, but not all, of the amino acid sequence of a naturally-occurring protein or polypeptide from which it is derived, e.g., MSF. The tribonectin may contain a polypeptide, the amino acid sequence of which is at least 50% identical to the sequence of residues 200-1140, inclusive, of SEQ ID NO: 1 (see Table 1), e.g., it contains the amino acid sequence of residues 200-1140, inclusive, of SEQ ID NO: 1. In another example, the polypeptide contains an amino acid sequence that is at least 50% identical to the sequence of residues 200-1167, inclusive, of SEQ ID NO: 1, e.g., one having the amino acid sequence identical to residues 200-1167, inclusive, of SEQ ID NO: 1. The polypeptide contains an amino acid sequence that is at least 50% identical to the sequence of residues 200-1212, inclusive, of SEQ ID NO:1, e.g., the amino acid sequence of residues 200-1212, inclusive, of SEQ ID NO: 1, or the polypeptide contains an amino acid sequence that is at least 50% identical to the sequence of residues 200-1263, inclusive, of SEQ ID NO: 1, e.g., an amino acid sequence identical to residues 200-1263, inclusive, of SEQ ID NO: 1. Preferably, the sequence of the polypeptide lacks the amino acid sequence of residues 1-24, inclusive, of SEQ ID NO: 1 and/or the amino acid sequence of residues 67-104, inclusive of SEQ ID NO:1.

The term “about” is used herein to mean a value that is +10% of the recited value.

### BRIEF DESCRIPTION OF THE DRAWINGS

FIG. 1 is a graph showing the offset between bovine synovial fluid (BSF) and 4:1 distilled water/glycerin solution in a multiple particle tracking microrheology experiment in which the time dependent ensemble-averaged mean squared displacement [as represented by D(τ) (m²/s)] is plotted vs. time.

FIG. 2 is a graph showing dilution studies of BSF with a 4:1 distilled water/glycerin solution in a multiple particle tracking microrheology experiment in which the time dependent ensemble-averaged mean squared displacement [as represented by D(τ) (m²/s)] is plotted vs. time. As BSF goes from a semidiluted to a diluted solution the concentration of the hydrophilic hyaluronic acid is lowered to the point (˜25% BSF by vol.) where network formation is hindered, rendering the fluid behavior increasingly Newtonian.

FIG. 3 is a graph showing the effects of enzymatic treatment with trypsin on BSF in a multiple particle tracking microrheology experiment in which the time dependent ensemble-averaged mean squared displacement [as represented by D(τ) (m²/s)] is plotted vs. time. A loss of particle entrapment ability of the network at low time-lags (<300 ms) is observed. The offset of the trypsinized BSF also shows a decrease in viscosity.

FIG. 4 is a graph showing the time-dependent ensemble average diffusion coefficient D(τ) of bovine synovial fluid (BSF), 4:1 glycerol/distilled water, trypsinized BSF and synovial fluid from a human patient with camptodactyly-arthropathy-coxa vara-pericarditis syndrome (CACP) in a multiple particle tracking microrheology experiment in which the time dependent ensemble-averaged mean squared displacement [as represented by D(τ) (m²/s)] is plotted vs. time.

FIGS. 5A-5D are charts showing structural heterogeneity of bovine synovial fluid (BSF)(FIG. 5A), 4:1 glycerol/distilled water (FIG. 5B), trypsinized BSF (FIG. 5C), and synovial fluid from a human patient with camptodactyly-arthropathy-coxa vara-pericarditis syndrome (CACP) (FIG. 5D) by looking at the time-dependent distribution of the MSD for individual particles.

FIG. 6 is a graph showing complex modulus for bovine synovial fluid (BSF), 4:1 glycerol/distilled water, trypsinized BSF and synovial fluid from a human patient with camptodactyly-arthropathy-coxa vara-pericarditis syndrome (CACP), as obtained from the time-dependent ensemble-average MSD. The x axis is the frequency (ω) in per seconds.

FIGS. 7a through 7d are graphs showing the viscoelastic behavior of glycerol, bovine synovial fluid (BSF), trypsinized BSF, and synovial fluid from a human patient with camptodactyly-arthropathy-coxa vara-pericarditis syndrome, respectively.

### DETAILED DESCRIPTION

The invention provides viscosupplementation compositions that can be administered to treat or prevent damage to joints. The addition of a tribonectin, such as lubricin, to viscosupplement preparations modifies its mechanical properties and permits manufacturers to more closely approximate the normal viscoelastic properties of synovial fluid. Healthy synovial fluid has an elasticity of 117 Pa and a viscosity of 45 Pa. The molecular weight of hyaluronate in synovial fluid is 6 million Da. A product like SYNVISC® (hylan), a hyaluronic acid preparation, mirrors many of these values in the absence of a tribonectin due to cross-linking of hyaluronate polymers. More preferable, though, would be viscosupplements containing hyaluronate having a greater molecule weight, which would improve several characteristics of the viscosupplement preparation, such as its tissue hydration properties, synovial cavity lubricating abilities, and residence time in the synovial cavity. The difficulty in using higher molecular weight hyaluronate in the viscosupplement preparations is that the use of high molecular weight hyaluronate polymers produces a hydrogel that exhibits plastic behavior (e.g., increased viscosity with a concomitant reduction in elasticity), which prevents ease of clinical use. The compositions of the present invention, in contrast, which are prepared by co-introducing a tribonectin, such as lubricin, into hyaluronate-containing viscosupplement preparations, exhibit improved viscosity and elasticity properties. The presence of a tribonectin in these hyaluronate hydrogels results in a viscosupplement preparation that exhibits increased residence time in the synovial cavity (due to, e.g., increased elastic properties) and reduced viscosity, which facilitates intra-articular administration. Similarly, other hyaluronate preparations, which do not have as high a molecular weight, but have elasticity and viscosity of about 60 and about 46 Pa·s respectively, can be engineered to increase their molecular weight and incorporated into a viscosupplement preparation containing a tribonectin, thereby maintaining the elasticity and viscosity parameters in a desirable range.

Thus, an aspect of the present invention is a composition that is prepared by adding a tribonectin to a hyaluronate-containing viscosupplement preparation, which increases the elasticity of the hyaluronic acid-containing preparation while also reducing its viscosity. The improved properties of the viscosupplement preparations of the present invention occur as a result of additional cross-links formed between the tribonectin and the hyaluronate polymers. This structure is in lieu of, or in addition to, the chemical cross-links which are created in modern viscosupplements. These properties are important to the normal properties of synovial fluid, which possesses a certain amount of elasticity that, by itself, plays a role in chondroprotection. The beneficial elasticity and viscosity properties imparted to viscosupplement preparations by the addition of a tribonectin are distinct from other beneficial properties imparted to viscosupplement preparations by the addition of a tribonectin, such as their ability to bind to and lubricate synovial cavity surfaces. Due to the interaction of a tribonectin with hyaluronate, and the surface activity of a tribonectin, which results in its binding to the cartilage surface, the net effect is to link hyaluronate to the surface of articular cartilage. In this manner, hyaluronate displays a more direct chrondroprotecting role and functions in its own right as a lubricant, which has been observed when hyaluronate is covalently bound to a surface (see, e.g., Tadmor et al., Macromolecules 36:9519-9526, 2003).

Hyaluronic Acid for use in the Compositions of the Invention

Synovial fluid is a semi-dilute solution of hyaluronate (HA) with additional constituents that play a wide variety of biological roles, which may include the regulation of the molecular structure of the fluid. Hyaluronic acid is a naturally-occurring polysaccharide containing alternating N-acetyl-D-glucosamine and D-glucuronic acid monosaccharide units linked with beta 1-4 bonds and the disaccharide units linked with beta 1-3 glycoside bonds with molecular weight range of about 50,000 to 8×10⁶. Synovial hyaluronate is a long linear negatively charged polyelectrolyte molecule with rotational bonds, usually occurring as the sodium salt (sodium hyaluronate). Intra-articular (injection) administration of high-molecular-weight HA to the patients is described as an effective procedure in the treatment of traumatized arthritic joints (Kikuchi et al., Osteoarthritis and Cartilage 4:99, 1996). The average molecular weight of synovial HA of healthy humans lies in the range (1.6-10.9)×10⁶ Da; while its concentration equals 2˜4 mg/mL (Balazs et al., Arthritis Rheum. 10:357, 1967). Molecular weight values of commercially available HA preparations obtained from various (natural) sources such as, e.g., bacteria Streptococcus zooepidemicus or Streptococcus equii, rooster combs, etc., vary in the range from hundreds of thousands to ca. 1-2 million Da. High-molecular-weight HA binds up to 1000 times more water than is its own mass and forms pseudoplastic, elastoviscous solutions, that behave as soft gels that reveal so-called shear-dependent viscosity and frequency-dependent elasticity (Larsen and Balazs, Adv. Drug Delivery Rev. 7:279, 1991). At the low magnitude of the shear tension, solutions of high-molecular-weight HA reveal high viscosity and low elasticity; while at the increasing values of shear tension the solutions become more elastic (Simon, Osteoarthritis 25:345, 1999). Such non-Newtonian behavior of synovial fluid is essential for the lubrication of joints during the (fast) movement. The cartilage surface is covered by a thin film of SF that smoothens (fine) unevenness of the articular structure. Deficiency of this layer leads to increased friction coefficient between the moving parts of the joint which results in strong pain (Nishimura et al., Biochim. Biophys. Acta 1380:1, 1998). Ultrapure (ready for injection application) preparations of the elastoviscous solutions of the hyaluronan sodium salt (HEALON™; Pharmacia, Uppsala, Sweden), obtained from the rooster combs, have found extended application especially in opthalmology (viscosurgery) (Nimrod et al, J. Ocular Pharmacol. 8:161, 1992], as well as in rheumatology (viscosupplementation) (Peyron, J. Rheumatology 20 Suppl. 39:10, 1993; T. Kikuchi et al, Osteoarthritis and Cartilage 4:99, 1996).

Recently another preparation for the intra-articular administration to OA patients was approved in the USA and some other countries. This product named HYLAN™ (Biomatrix Inc., Ridgefield, N.J., USA), contains high-molecular-weight HA originating from the rooster combs, and includes additionally cross-linked HA (L. S. Simon, Osteoarthritis 25:345, 1999). The water-soluble HYLANs with ultra-high molecular weight (on average around 6×10⁶ Da) that were prepared by chemical cross-linking of HA with formaldehyde reveal a significantly longer biological half-life period (Simon, Osteoarthritis 25:345, 1999). See also Larsen and Balazs, Adv. Drug Delivery Rev. 7:279, 1991; Al-Assafet al, Radiat. Phys. Chem. 46:207, 1995; and Wobig et al., Clin. Ther. 20:41, 19980 for summaries of pre-clinical and clinical trials involving injections of HYLAN™ solutions. Other HA-based viscosupplements are known as, HYLAGEL™, HYALGAN™, ARTZ™, SUPLASYN™, BIOHY™, ORTHOVISC™, and SYNVISC™. As used herein, the term hyaluronic acid, abbreviated as HA, means hyaluronic acid, a cross-linked form of HA, or its salts, such as, for example, sodium hyaluronate, potassium hyaluronate, magnesium hyaluronate, and calcium hyaluronate.

Hyaluronic acid was once thought to add viscoelastic effects to synovial fluid to enable hydrodynamic lubrication during periods of fast joint reciprocation. Under these circumstances some of the load from locomotion is borne by wedges of fluid between the articular surfaces. This effect restores ‘shock absorber’ characteristics to the diseased synovial fluid. Naturally occurring hyaluronate from human umbilical cord and rooster comb were used. Transformation into hylans was performed by cross-linking hydroxyl groups creating high molecular weight polymer networks (Pelletier and Martel-Pelletier, J Rheumatol (suppl 39)20:19-24, 1993.

An unintended consequence of meshed polymers is the creation of excluded volume which inhibit small molecule movement. For example, a 0.3 mg/mL solution of cross-linked hyaluronate requires 1 liter of aqueous solvent in order to be fully solvated. This concentration is 10 times less than normal synovial hyaluronate concentration, with the result that, in synovial fluid, each HA polymer is touching another. Understandably, injection of 0.3 mg of a viscosupplement into a confined knee joint would have significant effects on the rheological properties of a patient's synovial fluid and arrest small molecule movement by utilization of all available solvent. For example, solvation requirements would exclude cytokines and nociceptive mediators from triggering pain while restoring viscoelasticity.

Chondroprotection is served by a very different mechanism in synovial fluid. Synovial fluid is present to provide for lubrication of apposed and pressurized cartilaginous surfaces and to also nourish chondrocytes, as these highly specialized cells have no supportive blood supply. Digesting synovial fluid with hyaluronidase results in a non-viscous fluid which continues to lubricate (McCutchen, Wear 5:412-15, 1962). Synovial fluid digested with trypsin results in a viscous fluid which fails to lubricate (McCutchen, Fed Proc Fed Am Soc Exp Bio 25:1061-68, 1966 and Jay, Conn Tiss Re 28:71-88, 1992). The phenomenon of lubricating in the absence of viscosity is termed “boundary lubrication.”

The modicum of therapeutic value in the intra-articular administration of viscosupplements may be appropriate for those patients unable to tolerate NSAIDs (Lo et al., J. Am. Med. Assoc. 290:3115-21, 2003). However, the routine use of these devices in treating OA effectively is not well established as their mechanism of action is unclear. Multiple injections are required and therapeutic value is typically not seen until 3-6 months later, but can last longer than intra-articular steroid administration (Caborn et al., J. Rheumatol. 31:333-43. It should be appreciated that the HA-based viscosupplements that are currently commercially available are not articular lubricants and more likely work as retardants of pro-inflammatory factors. This effect may be more pronounced as the molecular weight of the hyaluronate is increased.

The human joint disease group most closely aligned with race horses that are treated with viscosupplements are active patients with inflammatory joint conditions (Vad et al., Sports Medicine 32:729-39, 2002) and not those with advanced OA. Deficient lubricating ability among patients with synovitis stands paradoxically in contrast to synovial fluid aspirated from joints of patients afflicted with OA (Jay et al., J. Rheumatol 31:557-64, 2004). These former patients demonstrate absent lubricating ability. By contrast, patients with OA have normal lubricating ability. These intriguing observations are partly explained by the fact that the lubricating moiety is produced by superficial zone articular chondrocytes (Flannery et al. Biochem. Biophys. Res. Comm. 234:535-41, 1999) and synovial fibroblasts (Jay et al., J. Rheumatol. 27:594-600, 2000), secreting superficial zone protein (SZP) and lubricin respectively. Both are highly homologous protein products of megakaryocyte stimulating factor gene expression. Patients with advanced OA undoubtedly may lack superficial zone chondrocytes and yet continue to have normal synovial fluid lubricating ability, suggesting that the synovial fibroblast contribution continues.

Disease states such as traumatic synovitis and RA, exemplified by synovial fluid deficient in lubricating ability, have both cell types affected. The histopathologic appearance of traumatic synovitis is similar to RA but less intense and extensive (Brit. J. Rheum.; 29:422-25, 1990). Inflammatory processes can lead to IL-1α expression which in the case of superficial zone articular chondrocytes, down regulates expression of SZP/lubricin and can ultimately lead to proteolysis. Arresting this process while at the same time restoring some of the mechanical features of synovial fluid (even the viscoelasticity by itself) may be of some importance. The implication is that an unlubricated joint will result in cartilage injury and premature wear, consequently leading to the fibrillation of cartilage and appearance OA.

The rheology of hyaluronate depends on aggregates and proteins present in the fluid (see Gribbon et al., Biochem. 350:329-35, 2000; Krause et al. Biomacromol. 2:65-9, 2001; and Pelletier et al., J. Biomed. Res. 54:102-8, 2001). The transport of nutrients and factors is greatly influenced by the molecular structure of the fluid. As noted above, two products of the gene PRG4, lubricin expressed by synovial fibroblasts (Jay et al., J. Rheum. 27:594-600, 2000) and superficial zone protein expressed by surface chondrocytes (Jay et al., J. Orthop. Res. 19:9-19, 2001; Flannery et al. Biochem. Biophys. Res. Comm. 234:535-41, 1999) participate in the boundary lubrication of cartilaginous joints. Hyaluronate and lubricin synergistically reduce friction under high loads, although hyaluronate alone does not have lubrication ability (Jay et al., Conn. Tiss. Res. 28:245-55, 1992).

Tribonectins for use in the Compositions of the Invention

Tribonectin, similar to proteoglycan 4 (PRG4), articular cartilage superficial zone protein (SZP), megakaryocyte stimulating factor precursor, or lubricin (Ikegawa et al., Cytogenet. Cell. Genet. 90:291-297, 2000; Schumacher et al., Arch. Biochem. Biophys. 311:144-152, 1994; Jay and Cha, J. Rheumatol., 26:2454-2457, 1999; and Jay, WIPO Int. Pub. No. WO 00/64930) is a mucinous glycoprotein found in the synovial fluid (Swann et al., J. Biol. Chem. 256:5921-5925, 1981). The amino acid sequence of MSF (SEQ ID NO: 1) is shown in Table 1. The gene encoding naturally-occurring full length MSF (SEQ ID NO:2) contains 12 exons, and the naturally-occurring MSF gene product contains 1404 amino acids with multiple polypeptide sequence homologies to vitronectin including hemopexin-like and somatomedin-like regions. Centrally-located exon 6 contains 940 residues and encodes a O-glycosylated mucin domain. A polypeptide encoded by nucleotides 631-3453 of SEQ ID NO:2 provides boundary lubrication of articular cartilage.

Tribonectin provides boundary lubrication of congruent articular surfaces under conditions of high contact pressure and near zero sliding speed (Jay et al., J. Orthop. Res. 19:677-87, 2001). These lubricating properties have also been demonstrated in vitro (Jay, Connect. Tissue Res. 28:71-88, 1992). Cells capable of synthesizing tribonectin have been found in synovial tissue and within the superficial zone of articular cartilage within diarthrodial joints (Jay et al., J. Rheumatol. 27:594-600, 2000).

In U.S. Pat. No. 6,743,774 and in U.S. patent application Ser. Nos. 09/897,188 and 10/038,694 are described methods of promoting lubrication between two juxtaposed biological surfaces using tribonectin, or fragments thereof. In PCT Publication No. WO 00/64930 are described tribonectin analogs and methods for lubricating a mammalian joint.

TABLE 1
MSF amino acid sequence (SEQ ID NO:1)

MAWKTLPIYLLLLLSVFVIQQVSSQDLSSCAGRCGEGYSRDATCNCDYNC
QHYMECCPDFKRVCTAELSCKGRCFESFERGRECDCDAQCKKYDKCCPDY
ESFCAEVHNPTSPPSSKKAPPPSGASQTIKSTTKRSPKPPNKKKTKKVIE
SEEITEEHSVSENQESSSSSSSSSSSSTIWKIKSSKNSAANRELQKKLKV
KDNKKNRTKKKPTPKPPVVDEAGSGLDNGDFKVTTPDTSTTQHNKVSTSP
KITTAKPINPRPSLPPNSDTSKETSLTVNKETTVETKETTTTNKQTSTDG
KEKTTSAKETQSIEKTSAKDLAPTSKVLAKPTPKAETTTKGPALTTPKEP
TPTTPKEPASTTPKEPTPTTIKSAPTTPKEPAPTTTKSAPTTPKEPAPTT
TKEPAPTTPKEPAPTTTKEPAPTTTKSAPTTPKEPAPTTPKKPAPTTPKE
PAPTTPKEPTPTTPKEPAPTTKEPAPTTPKEPAPTAPKKPAPTTPKEPAP
TTPKEPAPTTTKEPSPTTPKEPAPTTTKSAPTTTKEPAPTTTKSAPTTPK
EPSPTTTKEPAPTTPKEPAPTTPKKPAPTTPKEPAPTTPKEPAPTTTKKP
APTAPKEPAPTTPKETAPTTPKKLTPTTPEKLAPTTPEKPAPTTPEELAP
TTPEEPTPTTPEEPAPTTPKAAAPNTPKEPAPTTPKEPAPTTPKEPAPTT
PKETAPTTPKGTAPTTLKEPAPTTPKKPAPKELAPTTTKEPTSTTSDKPA
PTTPKGTAPTTPKEPAPTTPKEPAPTTPKGTAPTTLKEPAPTTPKKPAPK
ELAPTTTKGPTSTTSDKPAPTTPKETAPTTPKEPAPTTPKKPAPTTPETP
PPTTSEVSTPTTTKEPTTIHKSPDESTPELSAEPTPKALENSPKEPGVPT
TKTPAATKPEMTTTAKDKTTERDLRTTPETTTAAPKMTKETATTTEKTTE
SKITATTTQVTSTTTQDTTPFKITTLKTTTLAPKVTTTKKTITTTEIMNK
PEETAKPKDRATNSKATTPKPQKPTKAPKKPTSTKKPKTMPRVRKPKTTP
TPRKMTSTMPELNPTSRIAEAMLOTTTRPNQTPNSKLVEVNPKSEDAGGA
EGETPHMLLRPHVFMPEVTPDMDYLPRVPNQGIIINPMLSDETNICNGKP
VDGLTTLRNGTLVAFRGHYFWMLSPFSPPSPARRITEVWGIPSPIDTVFT
RCNCEGKTFFFKDSQYWRFTNDIKDAGYPKPIFKGFGGLTGQIVAALSTA
KYKNWPESVYFFKRGGSIQQYIYKQEPVQKCPGRRPALNYPVYGEMTQVR
RRRFERAIGPSQTHTIRIQYSPARLAYQDKGVLHNEVKVSILWRGLPNVV
TSAISLPNIRKPDGYDYYAFSKDQYYNIDVPSRTARAITTRSGQTLSKVW
YNCP

TABLE 2
MSF cDNA (SEQ ID NO:2)

| | |
|---|---|
| 1 | gcggccgcga ctattcggta cctgaaaaca acgatggcat |
| | ggaaaacact tcccatttac |
| 61 | ctgttgttgc tgctgtctgt tttcgtgatt cagcaagttt |
| | catctcaaga tttatcaagc |
| 121 | tgtgcaggga gatgtgggga agggtatcct agagatgcca |
| | cctgcaactg tgattataac |
| 181 | tgtcaacact acatggagtg ctgccctgat ttcaagagag |
| | tctgcactgc ggagctttcc |
| 241 | tgtaaaggcc gctgctttga gtccttcgag agagggaggg |
| | agtgtgactg cgacgcccaa |
| 301 | tgtaagaagt atgacaagtg ctgtcccgat tatgagagtt |
| | tctgtgcaga agtgcataat |
| 361 | cccacatcac caccatcttc aaagaaagca cctccacctt |
| | caggagcatc tcaaaccatc |
| 421 | aaatcaacaa ccaaacgttc acccaaacca ccaaacaaga |
| | agaagactaa gaaagttata |
| 481 | gaatcagagg aaataacaga agaacattct gtttctgaaa |
| | atcaagagtc ctcctcctcc |
| 541 | tcctcctctt cctcttcttc ttcaacaatt tggaaaatca |
| | agtcttccaa aaattcagct |
| | EXON 6 |
| 601 | gctaatagag aattacagaa gaaactcaaa gtaaaagata |
| | acaagaagaa cagaactaaa |
| 661 | aagaaaccta cccccaaacc accagttgta gatgaagctg |
| | gaagtggatt ggacaatggt |
| 721 | gacttcaagg tcacaactcc tgacacgtct accacccaac |
| | acaataaagt cagcacatct |
| 781 | cccaagatca caacagcaaa accaataaat cccagaccca |
| | gtcttccacc taattctgat |
| 841 | acatctaaag agacgtcttt gacagtgaat aaagagacaa |
| | cagttgaaac taaagaaact |
| 901 | actacaacaa ataaacagac ttcaactgat ggaaaagaga |
| | agactacttc cgctaaagag |
| 961 | acacaaagta tagagaaaac atctgctaaa gatttagcac |
| | ccacatctaa agtgctggct |
| 1021 | aaacctacac ccaaagctga aactacaacc aaaggccctg |
| | ctctcaccac tcccaaggag |
| 1081 | cccacgccca ccactcccaa ggagcctgca tctaccacac |
| | ccaaagagcc cacacctacc |
| 1141 | accatcaagt ctgcacccac cacccccaag gagcctgcac |
| | ccaccaccac caagtctgca |
| 1201 | cccaccactc ccaaggagcc tgcacccacc accaccaagg |
| | agcctgcacc caccactccc |
| 1261 | aaggagcctg cacccaccac caccaaggag cctgcaccca |
| | ccaccaccaa gtctgcaccc |
| 1321 | accactccca aggagcctgc acccaccacc cccaagaagc |
| | ctgccccaac tacccccaag |
| 1381 | gagcctgcac ccaccactcc caaggagcct acacccacca |
| | ctcccaagga gcctgcaccc |
| 1441 | accaccaagg agcctgcacc caccactccc aaagagcctg |
| | cacccactgc ccccaagaag |
| 1501 | cctgccccaa ctacccccaa ggagcctgca cccaccactc |
| | ccaaggagcc tgcacccacc |
| 1561 | accaccaagg agccttcacc caccactccc aaggagcctg |
| | cacccaccac caccaagtct |
| 1621 | gcacccacca ctaccaagga gcctgcaccc accactacca |
| | agtctgcacc caccactccc |
| 1681 | aaggagcctt cacccaccac caccaaggag cctgcaccca |
| | ccactcccaa ggagcctgca |
| 1741 | cccaccaccc ccaagaagcc tgccccaact acccccaagg |
| | agcctgcacc caccactccc |
| 1801 | aaggaacctg cacccaccac caccaagaag cctgcaccca |
| | ccgctcccaa agagcctgcc |
| 1861 | ccaactaccc ccaaggagac tgcacccacc acccccaaga |
| | agctcacgcc caccaccccc |
| 1921 | gagaagctcg cacccaccac ccctgagaag cccgcaccca |
| | ccacccctga ggagctcgca |
| 1981 | cccaccaccc ctgaggagcc cacacccacc acccctgagg |
| | agcctgctcc caccactccc |
| 2041 | aaggcagcgg ctcccaacac ccctaaggag cctgctccaa |
| | ctacccctaa ggagcctgct |
| 2101 | ccaactaccc ctaaggagcc tgctccaact acccctaagg |
| | agactgctcc aactacccct |
| 2161 | aaagggactg ctccaactac cctcaaggaa cctgcaccca |
| | ctactcccaa gaagcctgcc |
| 2221 | cccaaggagc ttgcacccac caccaccaag gagcccacat |
| | ccaccacctc tgacaagccc |
| 2281 | gctccaacta cccctaaggg gactgctcca actaccccta |
| | aggagcctgc tccaactacc |
| 2341 | cctaaggagc ctgctccaac tacccctaag gggactgctc |
| | caactaccct caaggaacct |
| 2401 | gcacccacta ctcccaagaa gcctgccccc aaggagcttg |
| | cacccaccac caccaagggg |
| 2461 | cccacatcca ccacctctga caagcctgct ccaactacac |
| | ctaaggagac tgctccaact |
| 2521 | acccccaagg agcctgcacc cactaccccc aagaagcctg |
| | ctccaactac tcctgagaca |
| 2581 | cctcctccaa ccacttcaga ggtctctact ccaactacca |
| | ccaaggagcc taccactatc |
| 2641 | cacaaaagcc ctgatgaatc aactcctgag ctttctgcag |
| | aacccacacc aaaagctctt |
| 2701 | gaaaacagtc ccaaggaacc tggtgtacct acaactaaga |
| | ctcctgcagc gactaaacct |
| 2761 | gaaatgacta caacagctaa agacaagaca acagaaagag |
| | acttacgtac tacacctgaa |
| 2821 | actacaactg ctgcacctaa gatgacaaaa gagacagcaa |
| | ctacaacaga aaaaactacc |
| 2881 | gaatccaaaa taacagctac aaccacacaa gtaacatcta |
| | ccacaactca agataccaca |
| 2941 | ccattcaaaa ttactactct taaaacaact actcttgcac |
| | ccaaagtaac tacaacaaaa |
| 3001 | aagacaatta ctaccactga gattatgaac aaacctgaag |
| | aaacagctaa accaaaagac |
| 3061 | agagctacta attctaaagc gacaactcct aaacctcaaa |
| | agccaaccaa agcacccaaa |
| 3121 | aaacccactt ctaccaaaaa gccaaaaaca atgcctagag |
| | tgagaaaacc aaagacgaca |
| 3181 | ccaactcccc gcaagatgac atcaacaatg ccagaattga |
| | accctacctc aagaatagca |
| 3241 | gaagccatgc tccaaaccac caccagacct aaccaaactc |
| | caaactccaa actagttgaa |
| 3301 | gtaaatccaa agagtgaaga tgcaggtggt gctgaaggag |
| | aaacacctca tatgcttctc |
| 3361 | aggccccatg tgttcatgcc tgaagttact cccgacatgg |
| | attacttacc gagagtaccc |
| 3421 | aatcaaggca ttatcatcaa tcccatgctt tccgatgaga |
| | ccaatatatg caatggtaag |
| 3481 | ccagtagatg gactgactac tttgcgcaat gggacattag |
| | ttgcattccg aggtcattat |
| 3541 | ttctggatgc taagtccatt cagtccacca tctccagctc |
| | gcagaattac tgaagtttgg |
| 3601 | ggtattcctt cccccattga tactgttttt actaggtgca |
| | actgtgaagg aaaaactttc |
| 3661 | ttctttaagg attctcagta ctggcgtttt accaatgata |
| | taaaagatgc agggtacccc |
| 3721 | aaaccaattc tcaaaggatt tggaggacta actggacaaa |
| | tagtggcagc gctttcaaca |
| 3781 | gctaaatata agaactggcc tgaatctgtg tattttttca |
| | agagaggtgg cagcattcag |
| 3841 | cagtatattt ataaacagga acctgtacag aagtgccctg |
| | gaagaaggcc tgctctaaat |
| 3901 | tatccagtgt atggagaaat gacacaggtt aggagacgtc |
| | gctttgaacg tgctatagga |
| 3961 | ccttctcaaa cacacaccat cagaattcaa tattcacctg |
| | ccagactggc ttatcaagac |
| 4021 | aaaggtgtcc ttcataatga agttaaagtg agtatactgt |
| | ggagaggact tccaaatgtg |
| 4081 | gttacctcag ctatatcact gcccaacatc agaaaacctg |
| | acggctatga ttactatgcc |
| 4141 | ttttctaaag atcaatacta taacattgat gtgcctagta |
| | gaacagcaag agcaattact |
| 4201 | actcgttctg ggcagacctt atccaaagtc tggtacaact |
| | gtccttagac tgatgagcaa |
| 4261 | aggaggagtc aactaatgaa gaaatgaata ataaattttg |
| | acactgaaaa acattttatt |
| 4321 | aataaagaat attgacatga gtataccagt ttatatataa |
| | aaatgttttt aaacttgaca |
| 4381 | atcattacac taaaacagat ttgataatct tattcacagt |
| | tgttattgtt tacagaccat |
| 4441 | ttaattaata tttcctctgt ttattcctcc tctccctccc |
| | attgcatggc tcacacctgt |
| 4501 | aaaagaaaaa agaatcaaat tgaatatatc ttttaagaat |
| | tcaaaactag tgtattcact |
| 4561 | taccctagtt cattataaaa aatatctagg cattgtggat |
| | ataaaactgt tgggtattct |
| 4621 | acaacttcaa tggaaattat tacaagcaga ttaatccctc |
| | tttttgtgac acaagtacaa |
| 4681 | tctaaaagtt atattggaaa acatggaaat attaaaattt |
| | tacactttta ctagctaaaa |
| 4741 | cataatcaca aagctttatc gtgttgtata aaaaaattaa |
| | caatataatg gcaataggta |
| 4801 | gagatacaac aaatgaatat aacactataa cacttcatat |
| | tttccaaatc ttaatttgga |
| 4861 | tttaaggaag aaatcaataa atataaaata taagcacata |
| | tttattatat atctaaggta |
| 4921 | tacaaatctg tctacatgaa gtttacagat tggtaaatat |
| | cacctgctca acatgtaatt |
| 4981 | atttaataaa actttggaac attaaaaaaa taaattggag |
| | gcttaaaaaa aaaaaaaaaa |
| 5041 | a |

TABLE 3

MSF Exon Boundaries

| | |
|---|---|
| Amino acid sequence | Nucleotide sequence in |

| | | |
|---|---|---|
| Exon | in SEQ ID NO:1 | SEQ ID NO:2 |

| | | |
|---|---|---|
| 1 | 1-24, inclusive | 34-105, inclusive |
| 2 | 25-66, inclusive | 106-231, inclusive |
| 3 | 67-104, inclusive | 232-345, inclusive |
| 4 | 105-155, inclusive | 346-498, inclusive |
| 5 | 156-199, inclusive | 499-630, inclusive |
| 6 | 200-1140, inclusive | 631-3453, inclusive |
| 7 | 1141-1167, inclusive | 3454-3534, inclusive |
| 8 | 1168-1212, inclusive | 3535-3670, inclusive |
| 9 | 1213-1263, inclusive | 3671-3822, inclusive |
| 10 | 1264-1331, inclusive | 3823-4026, inclusive |
| 11 | 1332-1371, inclusive | 4027-4146, inclusive |
| 12 | 1372-1404, inclusive | 4147-4245, inclusive |

The synovial fluid of an inflamed or injured joint contains proteolytic enzymes that degrade lubricating proteins or polypeptides. For example, infiltrating immune cells such as neutrophils secrete trypsin and/or elastase. Even a minor injury to an articulating joint or an inflammatory state can result in cellular infiltration and proteolytic enzyme secretion resulting in traumatic synovitis. Synovitis for a period of a few days or weeks can result in the loss of the cytoprotective layer of a joint, which in turn leads to the loss of cartilage. Non-lubricated cartilaginous bearings may experience premature wear which may initiate osteoarthritis. Individuals who clinically present with a traumatic effusion (e.g., “water on the knee”) are predisposed to developing osteoarthritis; the elaboration of proteolytic enzymes degrades and depletes naturally-occurring lubricating compositions in the synovial fluid. Depletion of natural lubricating compositions occurs in other inflammatory joint diseases such as rheumatoid arthritis. Replacing or supplementing the synovial fluid of such injured joints with the lubricating compositions of the invention prevents the development of osteoarthritis in the long term (e.g., years, even decades later) and immediately lubricates the joint to minimize short term damage.

Analogs, homologs, derivatives, or mimetics of tribonectins which are less susceptible to degradation in vivo can also be used in the present invention. Tribonectin analogs can differ from the naturally-occurring peptides by amino acid sequence, or by modifications which do not affect the sequence, or both. Modifications (which do not normally alter primary sequence) include in vivo or in vitro chemical derivatization of polypeptides, e.g., acetylation or carboxylation. Also included are modifications of glycosylation, e.g., those made by modifying the glycosylation patterns of the polypeptide during its synthesis and processing or in further processing steps, e.g., by exposing the polypeptide to enzymes which affect glycosylation, e.g., mammalian glycosylating or deglycosylating enzymes.

Where proteolytic degradation of the peptidyl component of a composition of the present invention following injection into the subject is a problem, replacement of a particularly sensitive peptide bond with a noncleavable peptide mimetic bond renders the resulting peptide more stable, and thus more useful as a therapeutic. To render the therapeutic peptidyl component less susceptible to cleavage by peptidases such as trypsin or elastase, the peptide bonds of a peptide may be replaced with an alternative type of covalent bond (a “peptide mimetic”). Trypsin, elastase, and other enzymes may be elaborated by infiltrating immune cells during joint inflammation. Trypsin cleaves a polypeptide bond on the carboxy-side of lysine and arginine; elastase cleaves on the carboxy-side of alanine, glycine. Thrombin, a serine protease which is present in hemorrhagic joints, cleaves a peptide bond on the carboxy-side of arginine. Collagenases are a family of enzymes produced by fibroblasts and chondrocytes when synovial metabolism is altered (e.g., during injury). These enzymes cut on the carboxy-side of glycine and proline. One or more peptidase-susceptible peptide bonds, e.g, those which appear in the KEPAPTT (SEQ ID NO:3) repeat sequence, can be altered (e.g., replaced with a non-peptide bond) to make the site less susceptible to cleavage, thus increasing the clinical half-life of the therapeutic formulation.

Such mimetics, and methods of incorporating them into polypeptides, are well known in the art. Similarly, the replacement of an L-amino acid residue with a D-amino acid is useful for rendering the a peptidyl component of a composition of the invention less sensitive to proteolysis. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl.

Also included as tribonectin peptide mimetics are tribonectin peptoid. Peptoids, or N-substituted glycines, are a specific subclass of peptidomimetics. They are closely related to their natural peptide counterparts, but differ chemically in that their sidechains are appended to nitrogen atoms along the molecule's backbone, rather than to the α-carbons (as they are in amino acids; see, e.g., Simon et al., P.N.A.S. USA 89:9367-9371, 1992). The tribonectin peptoids of the invention can be designed to mimetic any of the tribonectin polypeptide sequences, or fragments thereof, disclosed herein.

Clinical formulations of compositions of the present invention may also contain peptidase inhibitors such as N-methoxysuccinyl-Ala-Ala-Pro-Val chloromethylketone (an inhibitor of elastase). Other clinically acceptable protease inhibitors (e.g., as described in Berling et al., Int. J Pancreatology 24:9-17, 1998) such as leupeptin, aprotinin, α-1-antitrypsin, α-2-macroglobulin, α-1-protease inhibitor, antichymotrypsin (ACHY), secretory leukocyte protease inhibitor (PSTI) can also be co-administered with a composition of the invention to reduce proteolytic cleavage and increase clinical halflife. A cocktail of two or more protease inhibitors can also be coadministered.

Compositions of that include tribonectin polypeptides can be formulated in standard physiologically-compatible excipients known in the art., e.g., phosphate-buffered saline (PBS). Other formulations and methods for making-such formulations are well known and can be found in, e.g., Remington: The Science and Practice of Pharmacy (20th ed.), ed. A. R. Gennaro, Lippincott Williams & Wilkins, 2000, Philadelphia or Encyclopedia of Pharmaceutical Technology, eds. J. Swarbrick and J. C. Boylan, 2002, Marcel Dekker, New York).

Treatment using the Compositions of the Invention

The compositions of the invention can be administered to a mammal (e.g., a human) to treat soft tissue injuries, structural injuries, and degenerative or congenital conditions. In particular, the compositions of the invention can be administered to a mammal to treat or to alleviate, inhibit, or relieve the symptoms of osteoarthritis (which includes erosive osteoarthritis and is also known as osteoarthrosis or degenerative joint disease or DJD, or early osteoarthritis), rheumatoid arthritis, juvenile rheumatoid arthritis, spondyloarthropathies, gouty arthritis, infectious arthritis, structural joint defects (e.g., torn menisci and cruciate ligaments), synovitis (e.g., traumatic synovitis), and repetitive stress syndromes, as well as inflammation and symptoms associated with Sjogren's syndrome, Crohn's disease, and psoriatic arthritis, and systemic lupus erythematosus.

The compositions of the invention can also be administered to a mammal to alleviate, inhibit, relieve, or treat arthritic conditions associated with spondylitis, including ankylosing spondylitis, reactive arthritis (Reiter's syndrome), arthritis associated with chronic inflammatory bowel disease and AIDS-related seronegative spondyloarthropathy.

The compositions of the invention can also be administered to a mammal to alleviate or inhibit symptoms of rheumatic disease and disorders, e.g., systemic sclerosis and forms of scleroderma, polymyositis, dermatomyositis, necrotizing vasculitis and other vasculopathies, hypersensitivity vasculitis (including Henoch-Schonlein purpura), Wegener's granulomatosis, Giant cell arteritis, mucocutaneous lymph node syndrome (Kawasaki disease), Behcet's syndrome, Cryoglobulinemia, juvenile dermatomyositis, Sjogren's syndrome, overlap syndromes (includes mixed connective tissue disease), polymyalgia rheumatica, erythema nodosum, relapsing polychondritis, tendonitis (tenosynovitis), Bicipital tendenitis, bursitis, Olecranon bursitis, adhesive capsulitis of the shoulder (frozen shoulder) trigger finger, and Whipple's disease.

The compositions of the invention are also useful for alleviating or inhibiting the symptoms of diseases with rheumatic states, including, e.g., gout, pseudogout, chondrocalcinosis, amyloidosis, scurvy, specific enzyme deficiency states (including Fabry's disease, alkaptonuria, ochonosisi, Lesch-Nyhan syndrome, and Gaucher's disease), hyperlipoproteinemias (types II, Ia, IV), Ehlers-Danlos syndrome, Marfan's syndrome, pseudoxanthoma elasticum, and Wilson's disease.

The compositions of the invention can be administered to the joint (e.g., the knee, shoulder, wrist, ankle, or elbow) or connective tissue of a mammal.

It is envisioned that the compositions of the invention can be administered to treat connective tissue adhesions, e.g., adhesions in the hand or shoulder, and visceral adhesions, which occur after abdominal or thoracic surgery, connective tissue defects, e.g., trigger finger and carpal tunnel syndrome, as well as defects that occur following connective tissue grafts and transfers. The compositions of the invention can be administered to treat other common problems in which adhesion formation or restoration of tissue gliding is a problem.

Administration of Compositions of the Invention

Standard methods for delivery of the compositions of the invention can be used. Such methods are well known to those of ordinary skill in the art. For intra-articular administration, the tribonectin can be delivered in combination with the viscosupplement preparation containing HA or it can be delivered separately. In any event, the tribonectin is preferably administered at a concentration in the range of 20-500 μg/ml in a volume of approximately 0.1-2 ml per injection. For example, 1 ml of a composition containing a tribonectin (alone or with HA) at a concentration of 250 μg/ml is injected into a kneejoint using a fine (e.g., 14-22 gauge, preferably 18-22 gauge) needle. The compositions of the invention can also be administered by parenteral administration, such as by intravenous, subcutaneous, intramuscular, and intraperitoneal.

For prevention of surgical adhesions, the compositions of the invention containing a tribonectin and HA are administered in the form of gel, foam, fiber or fabric. A composition of the invention formulated in such a manner is placed over and between damaged or exposed tissue interfaces in order to prevent adhesion formation between apposing surfaces. To be effective, the gel or film must remain in place and prevent tissue contact for a long enough time so that when the gel finally disperses and the tissues do come into contact, they will no longer have a tendency to adhere. Compositions formulated for administration to inhibit or prevent adhesion formation (e.g, in the form of a membrane, fabric, foam, or gel) are evaluated for prevention of post-surgical adhesions in a rat cecal abrasion model (Goldberg et al., In Gynecologic Surgery and Adhesion Prevention. Willey-Liss, pp. 191-204, 1993). Compositions are placed around surgically abraded rat ceca, and compared to non-treated controls (animals whose ceca were abraded but did not receive any treatment). A reduction in the amount of adhesion formation in the rat model in the presence of the tribonectin- and HA-containing composition as compared to the amount of adhesion formation in the absence of the composition indicates that the composition is clinically effective to reduce tissue adhesion formation.

The compositions of the invention can also be used to coat artificial limbs and joints prior to implantation into a mammal. For example, such devices are dipped or bathed in a solution of a composition of the invention, e.g., as described in U.S. Pat. Nos. 5,709,020 and 5,702,456.

Lubricating polypeptides for use in the compositions of the invention are at least about 10 amino acids ((containing at least one KEPAPTT (SEQ ID NO:3)) or XXTTTX (SEQ ID NO:4) repeat), usually about 20 contiguous amino acids, preferably at least 40 contiguous amino acids, more preferably at least 50 contiguous amino acids, and most preferably at least about 60 to 80 contiguous amino acids in length. For example, the polypeptide is approximately 500 amino acids in length and contains 76 repeats of KEPAPTT (SEQ ID NO:3). The polypeptide is less than 1404 residues in length, e.g., it has the amino acid sequence of naturally-occurring MSF (SEQ ID NO: 1) but lacks at least 5, 10, 15, 20, or 24 amino acids at the N-terminus of naturally-occurring MSF. Such peptides are generated by methods known to those skilled in the art, including proteolytic cleavage of a recombinant MSF protein, de novo synthesis, or genetic engineering, e.g., cloning and expression of at least exon 6, 7, 8, and/or 9 of the MSF gene.

Tribonectin polypeptides for use in the compositions of the invention are also biochemically purified or isolated. The enzyme chymotrypsin cleaves at sites which bracket amino acids encoded by exon 6 of the MSF gene. Thus, a polypeptide containing amino acids encoded by exon 6 of the MSF gene (but not any other MSF exons) is prepared from a naturally-occurring or recombinantly produced MSF gene product by enzymatic digestion with chymotrypsin. The polypeptide is then subjected to standard biochemical purification methods to yield a substantially pure polypeptide suitable for therapeutic administration, evaluation of lubricating activity, or antibody production.

Therapeutic compositions are administered in a pharmaceutically acceptable carrier (e.g., physiological saline). Carriers are selected on the basis of mode and route of administration and standard pharmaceutical practice. A therapeutically effective amount of a therapeutic composition of the invention (e.g., one containing a lubricating polypeptide, such as lubricin) is an amount which is capable of producing a medically desirable result, e.g., boundary lubrication of a mammalian joint, in a treated animal. A medically desirable result is a reduction in pain (measured, e.g., using a visual analog pain scale described in Peyron et al., 1993, J. Rheumatol. 20 (suppl.39):10-15) or increased ability to move the joint (measured, e.g., using pedometry as described in Belcher et al., 1997, J. Orthop. Trauma 11:106-109). Another method to measure lubricity of synovial fluid after treatment is to reaspirate a small volume of synovial fluid from the affected joint and test the lubricating properties in vitro using a friction apparatus as described herein.

As is well known in the medical arts, dosage for any one animal depends on many factors, including the animal's size, body surface area, age, the particular compound to be administered, sex, time and route of administration, general health, and other drugs being administered concurrently. Administration is generally local to an injured or inflamed joint or connective tissue associated with a joint. Alternatively, a timed-release formulation of the composition of the invention can be prepared, in which one or more of the components (e.g., the HA or the tribonectin) are slowly released (e.g., released over the course of 1-5 hours, 1-24 hours, 1-2 days, or 1-2 weeks) at the site of an injured or inflamed joint or a connective tissue associated with a joint, following administration of the composition.

Veterinary Applications

Canine osteoarthritis is a prevalent clinical disorder that is treated using the methods described herein. Osteoarthritis afflicts an estimated one in five adult dogs; an estimated 8 million dogs suffer from this degenerative, potentially debilitating disease. Yet, many owners do not recognize the signs of chronic canine pain. While any dog can develop osteoarthritis, those most at risk are large breeds, geriatric dogs, very active dogs (such as working or sporting animals), and those with inherited joint abnormalities such as hip or elbow dysplasia.

Equine degenerative joint disease such as osteoarthritis is a cause of lameness and impaired performance in horses. As with humans and other mammals, degenerative joint diseases which affect horses are progressive disorders of synovial joints characterized by articular cartilage degeneration and joint effusion. Acute or chronic trauma, overuse, developmental disease, joint instability and old age leads to synovitis, impaired chondrocyte metabolism, and the formation of fissures in the joint cartilage. Destructive enzymes such as trypsin, elastase, stromelysin and hyaluronidase are released into the joint where they degrade synovial fluid and cartilage components, resulting in decreased synovial fluid viscosity, poor lubrication, depressed cartilage metabolism and enhanced wear resulting in pain and cartilage erosion. Current therapeutic approaches include medications for pain relief and anti-inflammatory drugs. The compositions and methods described herein are useful to replenish the lubricating capabilities of the affected joint.

Microrheology Studies

The effects of tribonectin upon synovial fluid's viscosity was studied in a novel multiple particle tracking technique which studies random walk behavior of particles introduced into synovial fluid. Viscosity is calculated from mean squared displacement (MSD) of tracked particles via the Einstein-Stokes relation. The advantage of this technique is that very small samples volumes are required; suitable for the study of clinical aspirates.

Experimental Setup

Fluorescent microspheres (Duke Scientific Corp., Palo Alto, Calif.) of 200 nm mean-diameter were added to the solutions being tested (0.3% volume fraction). A drop (˜2-5-μL) of the sample was deposited in a hydrophobic multi-well slide (Erie 30 Scientific, Portsmouth, N.H.). This static condition of the fluid was confirmed by observing the relative motion of tracers over an extended amount of time (t>20 s). The slide was covered and placed on the stage of an inverted light microscope, (Nikon TE 200) and a peltier chip (MELCOR, Trenton, N.J.) temperature set up was placed on top of the slide to stabilize the temperature (˜295 K). The temperature of the sample was set using a thermoelectric controller (Oven Industries, Mechanicsburg, Pa.), which varies the amount current through the chip. An objective (Nikon) of 100×, 1.4 NA was used for magnification. The fluorescent beads were tracked with a 1500-EX charged-coupled digital (CCD) camera (IDT, Tallahassee, Fla.) of 6.45 μm×6.45 Jwn pixel resolution and 12 bit of dynamic range with 1×1 binning, for an effective 64.5 nm×64.5 nm per pixel resolution for the optical system.

The solutions studied were:
1) Glycerol 99.5+% (Sigma-Aldrich, Milwaukee, Wis.), used for the control solution due to its Newtonian behavior and its viscosity of 1P, which is slightly higher than that of BSF;
2) Bovine synovial fluid (BSF) and BSF diluted with 4:1 glycerol, diluted with doubly-distilled water (DDW), to 1:1 (50% BSF) and 3:1 (75% BSF) solutions;
3) BSF digested with TPCK-treated trypsin (Worthington Biochemicals, Freehold, N.J.; and
4) Synovial fluid from a patient with camptodactyly-arthropathy-coxa vara-pericarditis syndrome (CACP).
   Multiple Particle Tracking Microrheology

Particles in different locations of the middle plane of athe sample were tracked separately and a region of interest (ROI) of approximately 8 μm×8 μm was used to confirm the same particle was being tracked frame after frame. The time-dependent ensemble-average mean squared displacement (MSD) of each particle was measured and analyzed over a ranged of frequencies using a MATLAB code. The code fits a Gaussian distribution to the Airy disks formed by the intensity of the light emitted from the flourophores in each particle. The center of this distribution is taken as the center of the particle and is tracked to measure the time-dependent mean-squared displacement (MSD). Subpixel interpolation is done and in this manner particles are tracked with ˜5 nm spatial resolution. To avoid particle-on-particle interaction artifacts, only particle probes with approximately ten diameters distance from the next probe were tracked. Approximately 80 particles were tracked for these experiments for a time of 12 s each at a rate of 16 Hz. The time-dependent MSD ensemble-average was used to study the time-dependent diffusivity of the tracers in the fluid preparations and in this way a description of the macroscopic behavior of the complex fluid was derived from microscopic measurements. The time-dependent ensemble-average diffusion coefficient was extracted from the two dimensional random walk model, using the formula I (Berg, Random Walks in Biology, Expanded Edition, Princeton University Press, pp. 5-12, Berg, 1993).
D(τ)= [symbol] [symbol] Δr²(τ) [symbol] [symbol] /4τ (1)

The structural and mechanical heterogeneity of the network in the dilute solutions was probed by observing the time-dependent distribution of MSD of individual particle at different locations throughout the sample (Apgar et al., Multiple-Particle Tracking Measurements of Heterogeneities in Solutions of Actin Filaments and Actin Bundles, Biophysical J. 79:1095-1106, 2000; Xu et al., Microheterogeneity and Microrheology of Wheat Gliadin Suspensions Studies by Multiple-Particle Tracking, Biomacromolecules 3:92-99, 2002). The time-dependent complex modulus |G*(ω)| along with its components, the elastic Gs(ω), and loss Gd(ω) moduli for the samples was calculated for the samples bywith the method described by Gardel et al., and developed by Mason et al. [5, 6], using formula 2, where k_{b} is the Boltzmann constant, T is the temperature in Kelvins, α is the radius of the probes, [symbol] Δr²(r) [symbol] is the MSD with respect to the frequency (c) of interest, r is the gamma function and d ln [symbol] Δr²(τ) [symbol] /d ln τ|_{τ=1/ω} is the slope of the MSD, [symbol] Δr²(τ) [symbol] , with respect to the time lag (τ) between measurements.

$$\vert {G}^{*} ( ω ) \vert \approx \frac{{k}_{b} T}{π a 〈 Δ {r}^{2} ( 1 / ω ) 〉 Γ [ 1 + ⅆ ln 〈 Δ {r}^{2} ( τ ) 〉 / ⅆ ln τ ]} \qquad ( 2 )$$

It is assumed that the fluid being probed is isotropic and incompressible around the sphere, which is acceptable at these low Re numbers. Also the characteristic mesh size of the network in the complex fluid is smaller that the diameter of the particle.

Results

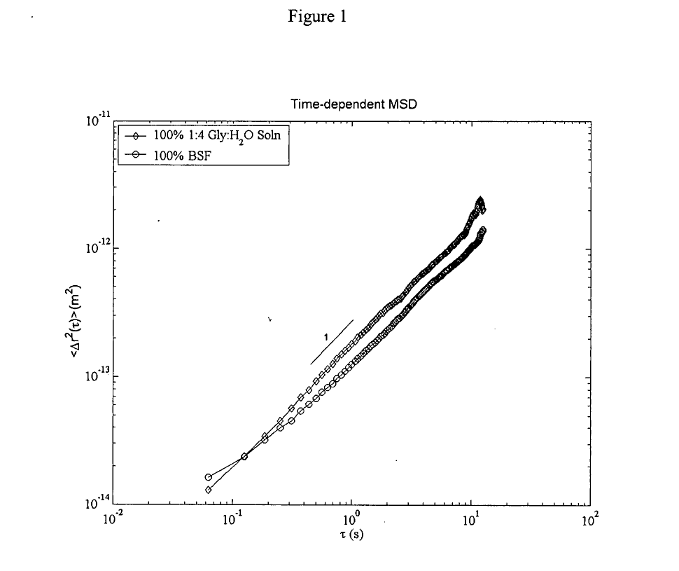

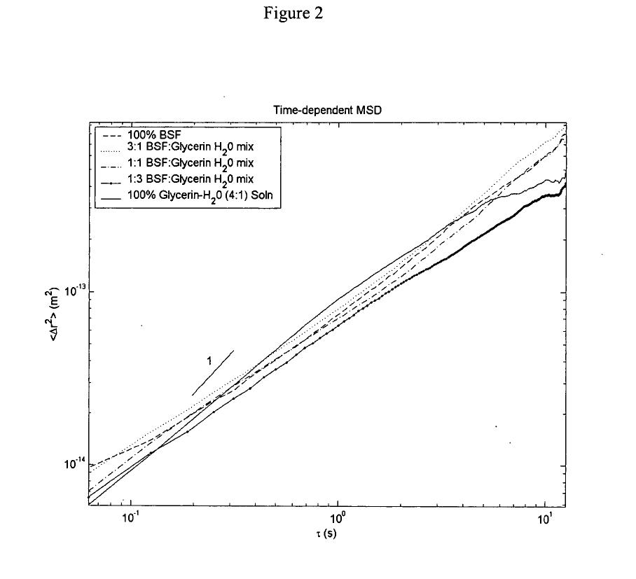

In order to test the system, BSF was compared to and subsequently diluted with a mixture of glycerol, a Newtonian fluid of known viscosity (1P), and DDIW. The time-dependent ensemble-average MSD of probes embedded in polymeric viscoelastic fluids adopts a power law, ( [symbol] Δr²(τ) [symbol] ˜τ^{α}), behavior, where α is the slope of the natural logarithmic curve. The slope over the range of time scales probed sheds light on the viscoelastic behavior of the fluid. A 4:1 glycerol to DDIW (GDDIW) solution was used in this experiment to match the viscous behavior of BSF at lower frequencies. At these frequencies BSF shows a mostly diffusive behavior which is evident by the slope (α≈1), of the time-dependent ensemble-average MSD. As shown in FIG. 1, the offset between the BSF and 4:1 GDDIW solution curves shows the slight differences in viscosity (˜75 cP). At low time-lags (<300 ms) BSF shows a subdiffusive behavior (α<1) due to particle entrapment in the hyaluronic acid (HA) network. Shown in FIG. 2 are subsequent dilutions of BSF with the 4:1 GDDIW solution, where the slope (a) of the time-dependent MSD goes from subdiffusive (α<1) to diffusive (a 1) behavior at low time-lags (<300 ms). As BSF goes from a semidiluted to a diluted solution the concentration of the hydrophilic hyaluronic acid is lowered to the point (˜25% BSF by vol.) where network formation is hindered, rendering the fluid behavior increasingly Newtonian. All of the BSF-glycerol mixture solutions exhibit a subdiffusive behavior at higher time-lags although to a lesser extend than that for lower time-lags. This relationship of concentration and viscoelasticity was also shown by Xu, et al., vide supra, in wheat gliadin suspensions.

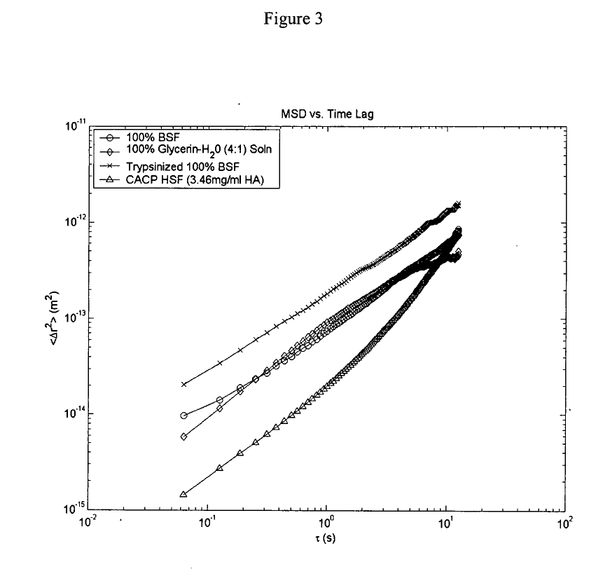

Apgar et al., vide supra, demonstrated the effects of regulatory protein on the network formation and overall viscoelasticity of complex fluids. Similarly, the influence of Purified Synovial Lubricating Factor (PSLF) on the structural and mechanical properties of Synovial Fluid were studied using multiple-particle-tracking microrheology (MPTM). In FIG. 3 the effects of enzymatic treatment with trypsin on BSF are shown with the loss of particle entrapment ability of the network at low time-lags (<300 ms). It is likely that the elastic effects in the network were shifted to even lower time-lags. Also the offset of the trypsinized BSF shows a decrease in viscosity. This change in viscosity may be due to the enzymatic digestion of the bulk of the proteins in the BSF. Therefore, it was important to study the microrheology of synovial fluid that was only missing PSLF, while having the other macromolecules that exist in normal synovial fluid. To accomplish this, camptodactyly-arthropathy-coxa vara-pericarditis syndrome (CACP) synovial fluid with a hyaluronic acid (HA) concentration of 3.46 mg/mL was studied. This concentration of HA was the same for the other solutions of synovial fluid. In this manner the network forming molecule was maintained at a controlled concentration and any changes in network formation were due to the regulatory molecules. The amount of CACP synovial fluid was too small for the use of other techniques to study its bulk rheology. Not only MPTM is advantageous as the only tool available to probe the microenvironment of complex fluids, but it also allows for the study of scarce fluids since the amount needed is close to 100 μL. In FIG. 3 it is seen that CACP synovial fluid at low time-lags (<300 ms) exhibits the purely diffusive behavior of a Newtonian fluid with a slope close to unity. Enzyme-treated BSF and CACP-HSF exhibited a diffusive behavior at both low and high time lags for the same bead sized (220 nm). This behavior resembles that of 4:1 glycerol/water, a Newtonian fluid. The relaxation time for CACP-HSF was an order of magnitude higher than that of both the BSF and the ET-BSF solutions, and in the same range as a 4 mg/mL solution of umbilical-cord hyaluronate (UHA). Table 4 shows the relaxation times of different samples under oscillatory shear flow.

TABLE 4
Relaxation times of different samples of synovial
fluid under oscillatory shear flow

| | Sample | | | |
|---|---|---|---|---|
| | BSF | ET-BSF | CACP-HSF | UHA |
| Relaxation Times (ms) | 0.1-0.125 | 0.08-0.1 | 0.014-0.016 | 0.01-0.016 |

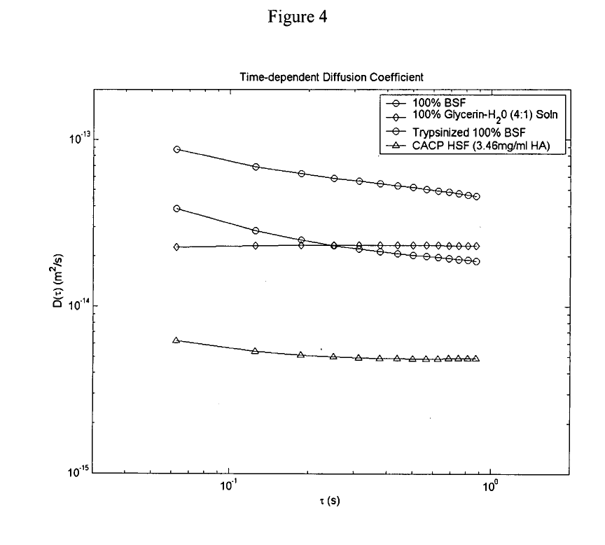

FIG. 4 depicts the time-dependent ensemble average diffusion coefficient D(τ) of the samples. Glycerol behaves as a Newtonian fluid with a constant diffusion coefficient over the range of time-lags. The synovial fluid samples had a time-dependent ensemble-average diffusion coefficient with a plateau at higher time-lags. The loss of overall viscosity after enzymatic digestion of the BSF is evident as the higher diffusion coefficient over the whole range of frequencies. In this fluid the probes were able to move with greater ease that the probes in the rest of the samples. It is evident by the low diffusion coefficient of the 200 nm probes in the CACP that its viscosity is much higher than that of normal BSF.

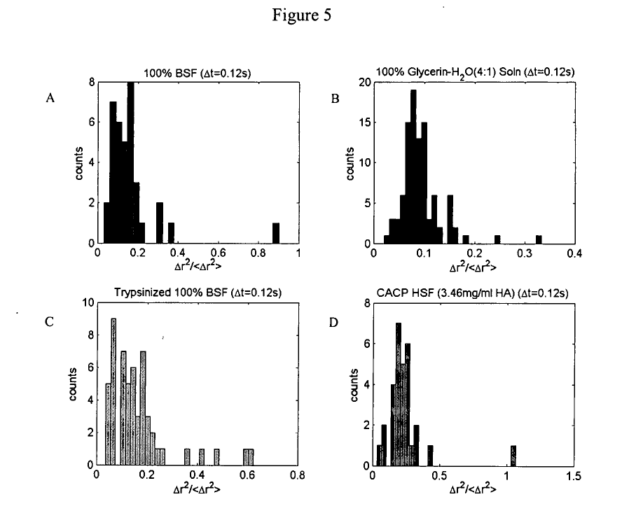

The structural heterogeneity of the networks was assessed by looking at the time-dependent distribution of the MSD for individual particles, as shown in FIG. 5. The distribution of the [symbol] Δr²(τ) [symbol] is measured at a time-lag of 0.12 s and normalized by the corresponding ensemble-averaged mean. As expected, the distribution of the [symbol] Δr²(τ) [symbol] of the glycerol solution was symmetric about the mean and exhibited the normal distribution of a Newtonian fluid. The other distributions show signs of deviating from this normal distribution, but more particles (˜250) at more locations across the sample need to be observed at different time-lags to make a better statistical assessment of heterogeneity of the complex fluids and how it changes under different conditions.

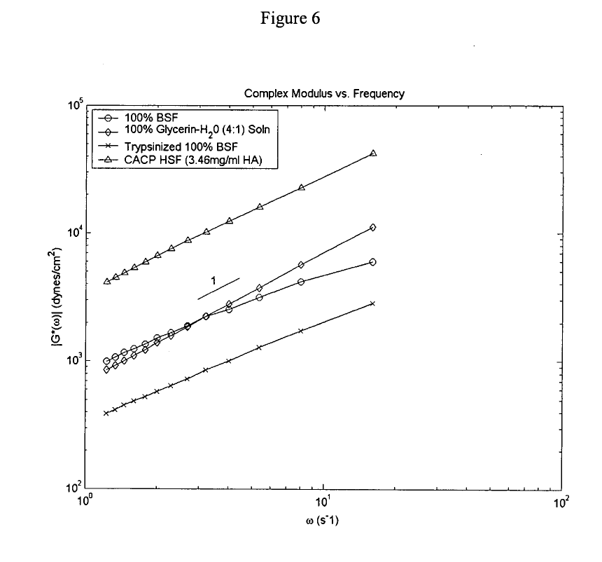

The complex modulus for the fluids tested, as shown in FIG. 6, was obtained from the time-dependent ensemble-average MSD. At low frequencies (ω) all of the fluids exhibit a purely diffusive behavior. The slope near unity is attributed to the low frequency viscosity associated with the relaxation of colloidal entanglements [6]. At higher frequencies (ω) BSF exhibits a decreasing slope that seems to approach a plateau region due to the network entanglement of the probes. The enzymatic treatment of BSF seems to have hindered its elasticity at higher frequencies, as the plateau region does not occur at the frequencies seen for BSF. The CACP synovial fluid also exhibits a slope near unity at these higher frequencies lacking the elastic behavior of normal BSF. It is important to recognized that this elastic behavior may be shifted to higher frequencies since the HA concentration is the same for all the synovial fluid preparation and that regulatory proteins may play a part in enhancing the viscoelasticity of the synovial fluid.

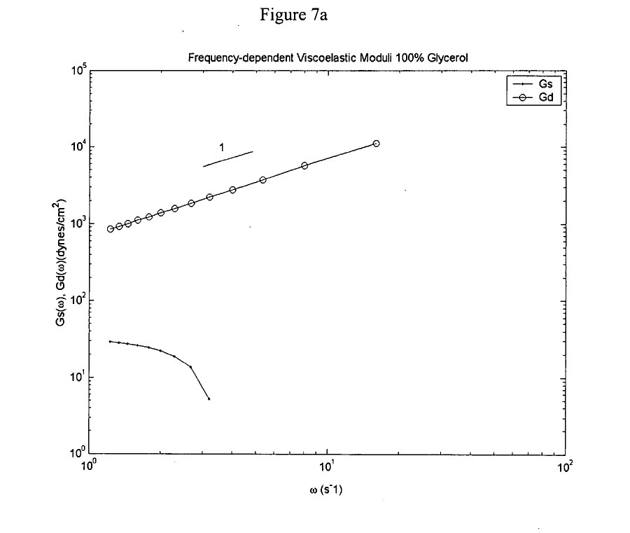

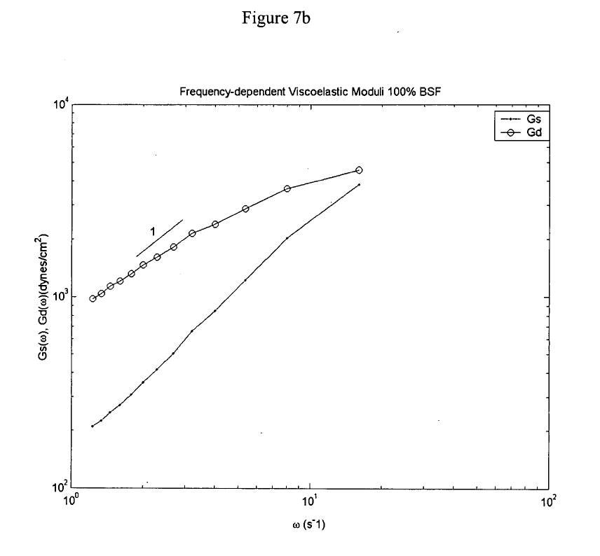

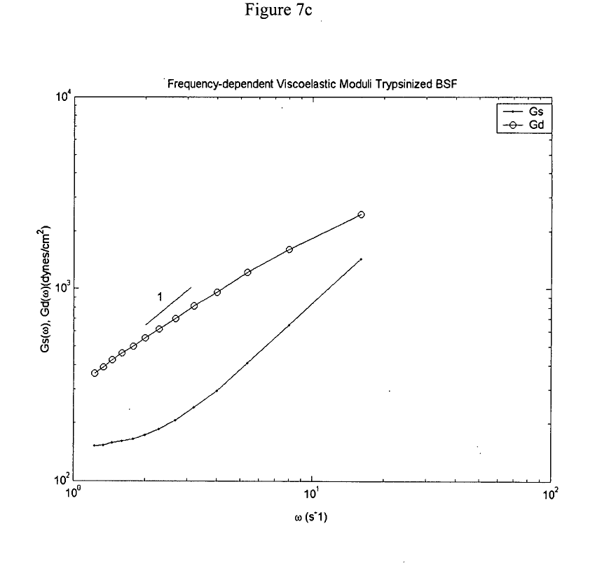

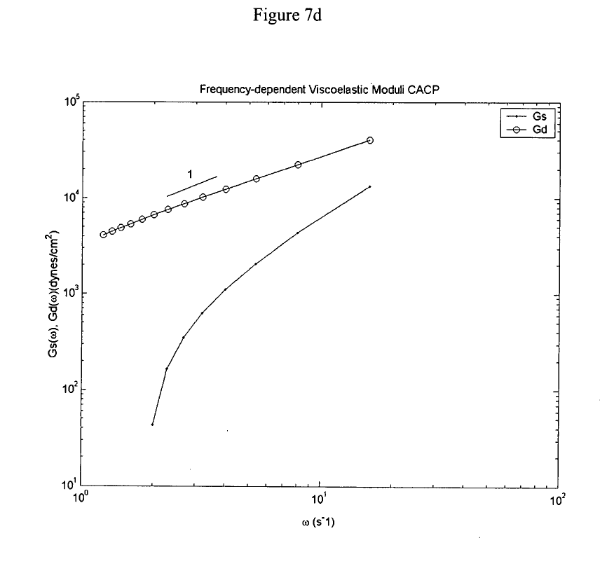

The viscoelastic behavior is shown in FIG. 7. As is seen from FIG. 7a, the glycerol solution exhibits a dominant dissipation modulus, of slope close to unity, at low and high frequencies (ω), which is expected for a Newtonian fluid. The presence of the much smaller storage modulus is due to small errors of measurement using the CCD camera. The synovial fluid solutions (FIGS. 7b, c, d) show the behavior expected for polymeric networks. At low frequencies (ω) the dissipation modulus dominates the behavior of the fluid, initially rising with a slope near unity and subsequently approaching a plateau, while at higher frequencies (ω), it is the storage modulus that dominates, which crosses over to higher values. There are still differences in the frequencies at which the cross-over of the moduli will occur. For BSF this cross over is at a lower frequency than that for the enzyme treated and CACP synovial fluid, pointing to an enhancement of viscoelasticity by regulatory proteins via network formation and organization.

All publications and patents cited in this specification are hereby incorporated by reference herein as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A viscosupplementation composition comprising hyaluronic acid at a concentration of from 0.1 mg/mL to 50.0 mg/mL and tribonectin at a concentration of from 0.1 μg/mL to 1.0 mg/mL.

2. The composition of claim 1, wherein said hyaluronic acid is at a concentration of from 2.5 mg/mL to 5.0 mg/mL.

3. The composition of claim 1, wherein said hyaluronic acid is at a concentration of from 3.0 mg/mL to 4.0 mg/mL.

4. The composition of claim 1, wherein said hyaluronic acid and said tribonectin are at a molar ratio of from 2:1 to 10:1.

5. The composition of claim 1, wherein said hyaluronic acid is cross-linked.

6. The composition of claim 1, wherein said tribonectin increases the elasticity of, and reduces the viscosity of, said composition.

7. A medicament comprising the composition of claim 1 for use in lubricating or chondroprotecting a mammalian joint.

8. The medicament of claim 7, wherein said joint is an articulating joint of a human.

9. The medicament of claim 7, wherein said joint is an articulating joint of a horse.

10. The medicament of claim 7, wherein said joint is an articulating joint of a dog.

11. The medicament of claim 7, wherein said composition is administered intra-articularly.

12. A medicament comprising a tribonectin admixed with a viscosupplement, wherein said medicament is prepared for use in the lubrication and chondroprotection of a mammalian joint, and wherein said tribonectin increases the elasticity of, and reduces the viscosity of, said viscosupplement.

13. The medicament of claim 12, wherein said tribonectin is added to a final concentration of from 0.1 μg/mL to 1.0 mg/mL.

14. The medicament of claim 12, wherein said viscosupplement comprises hyaluronic acid.

15. The medicament of claim 14, wherein said hyaluronic acid is present in said viscosupplement at a concentration of from 0.1 mg/mL to 50.0 mg/mL.

16. The medicament of claim 14, wherein the molar ratio of said tribonectin to said hyaluronic acid is from 2:1 to 10:1 after the addition of said tribonectin.

17. The medicament of claim 12, wherein said joint is an articulating joint of a human.

18. The medicament of claim 12, wherein said joint is an articulating joint of a horse.

19. The medicament of claim 12, wherein said joint is an articulating joint of a dog.

20. The medicament of claim 12, wherein said viscosupplement is administered intra-articularly to said joint after the addition of said tribonectin.

21. The medicament of claim 12, wherein said viscosupplement is administered intra-articularly to said joint before the addition of said tribonectin.

22. A method of treating or reducing the symptoms of a soft tissue injury, a structural injury, or a degenerative or congenital condition in a mammal in need thereof comprising administering the composition of claim 1 to said mammal.

23. The method of claim 22, wherein said composition is administered to treat or reduce the symptoms of osteoarthritis, synovitis, degenerative joint disease, rheumatoid arthritis, structural joint defects, or repetitive stress injury.

24. The method of claim 22, wherein said method comprises administering said composition to a joint of said mammal.

25. The method of claim 23, wherein said joint is an articulating joint.

26. The method of claim 22, wherein said joint is a knee, shoulder, wrist, ankle, or elbow.

27. The method of claim 22, wherein said mammal is human, horse, or dog.


SEQUENCE LISTING (4 sequences)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTAPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGEMTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - DNA, 5041 bp, Homo sapiens
gcggccgcgactattcggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaatttggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccgctcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaatgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaaaaaaaaaaaaaaaa

SEQ ID NO: 3 - protein, 7 aa, Homo sapiens
KEPAPTT

SEQ ID NO: 4 - protein, 6 aa, Homo sapiens
  misc_feature (1)..(2): Xaa can be any naturally occurring amino acid
  misc_feature (6)..(6): Xaa can be any naturally occurring amino acid
XXTTTX